(19) United States
(12) Patent Application Publication
(10) Pub. No.: US 2008/0077067 A1
(43) Pub. Date: Mar. 27, 2008

(54) Hygenic applicator

(76) Inventor: Joanna L. Mata, San Antonio, TX (US)

Correspondence Address: Galasso & Associates, LP, P.O. Box 26503, Austin, TX, 78755-0503, US

(21) Appl. No.: 11/525,382

(22) Filed: Sep. 22, 2006

(51) Int. Cl. A61M 35/00

(52) U.S. Cl. 604/2; 604/289

(57) ABSTRACT

A hygienic applicator for applying a cleaning solution to the umbilical cord area of a newborn. The hygienic applicator includes a support member that is substantially cylindrical in shape. The support member includes two ends. Superposed on each end is a tip made from an absorbent material that is designed to retain a desired cleaning solution and assist the user in applying the cleaning solution to the desired area. The support member is further enclosed in a container having a cavity of sufficient size to substantially enclose the support member therein and prevent the evaporation of the cleaning solution for the applicator. The container further includes an opening mechanism for facilitating the easy opening of the container.

### FIELD OF THE INVENTION

The present invention relates to a hygienic cleaning applicator, more specifically but not by way of limitation, a single applicator substantially enclosed and stored in a container having a portion thereof saturated with a desired cleaning solution that is to be used for cleaning the umbilical cord area of an infant.

### BACKGROUND

Thousands of newborns are born everyday. With each newborn, the parents accept great responsibilities. Newborns require extensive and constant care during their first few months. Everything from nourishment to hygienic needs is now the responsibility of the parents.

One specific issue with hygiene for newborns is bathing and proper cleaning of the newborn. A particular area of concern is the umbilical cord remnant. During the first few weeks the remains of the umbilical cord is still present on the newborn. This area is very prone to infection and great care must be taken when cleaning this area. This area adjacent to the umbilical cord must be cleaned frequently and properly in order to avoid infection.

One problem with cleaning the umbilical cord is accessing the right hygienic device that can be easily used by the caretaker. Typically, a conventional cotton applicator is used along with a disinfectant such as isopropyl alcohol solution. Unfortunately, the caretaker must endure the cumbersome task of trying to ensure the safety of the newborn while trying to prepare a cotton applicator with isopropyl alcohol. Attempting to absorb some isopropyl alcohol from a conventional bottle while steadying a moving child is extremely difficult.

Accordingly, there is a need for a hygienic applicator that is easily used and is pre-saturated with a conventional cleaning solution to assist in cleaning the umbilical cord area of a newborn.

### SUMMARY OF THE INVENTION

It is the object of the present invention to provide a hygienic applicator for use in cleaning the umbilical cord area of a newborn that is pre-saturated with a cleaning solution.

It is a further object of the present invention to provide a hygienic applicator for use in cleaning the umbilical cord area of a newborn that is pre-saturated with a cleaning solution that is enclosed in a flexible and non-permeable container.

Another object of the present invention is to provide a hygienic applicator for use in cleaning the umbilical cord area of a newborn that is pre-saturated with a cleaning solution that is relatively inexpensive.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawing wherein:

FIG. 1 is a perspective view of an embodiment of the present invention enclosed in a container; and

FIG. 2 is a perspective view of an embodiment of the present invention removed from its container.

### DETAILED DESCRIPTION

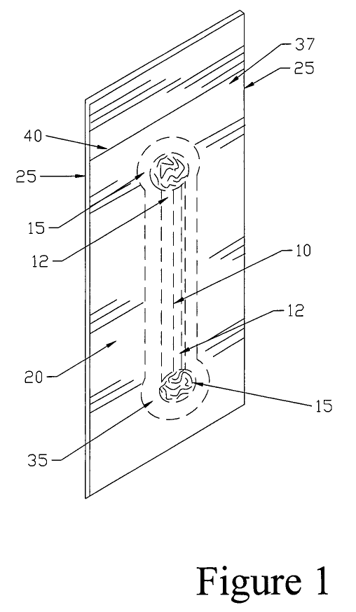

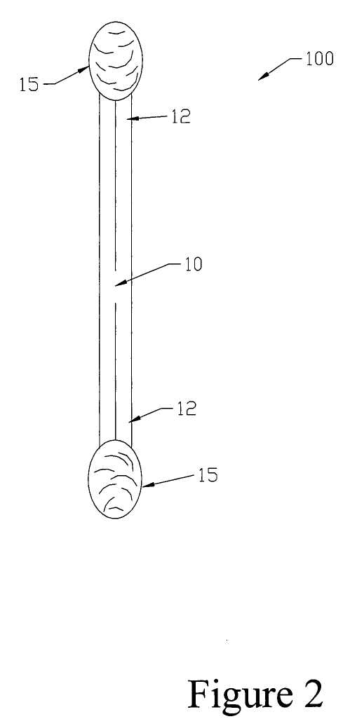

Referring now to the drawings submitted herewith wherein the various elements depicted therein are not necessarily drawn to scale and wherein like elements are identified with like reference numerals and in particular to FIGS. 1 and 2, there is illustrated a preferred embodiment of a hygienic applicator 100.

The hygienic applicator 100 comprises a support member 10 that is substantially cylindrical in shape and provides structural support for the hygienic applicator 100. The support member 10 is manufactured from a suitable durable material such as but not limited to plastic, wood or cardboard. It is contemplated within the scope of the present invention that the support member 10 is resilient so as to assist the user in preventing any injury to the desired area where the hygienic applicator 100 is utilized such as but not limited to the umbilical cord area of a newborn. Those skilled in the art will recognize that the hygienic applicator 100 could be utilized on numerous different objects or areas on the human body. Although no specific size is required for the support member 10, good results have been achieved with a support member 10 that is approximately four inches in length. Furthermore, it is contemplated within the scope of the present invention that the support member 10, rather than being cylindrical, could be numerous different shapes and still perform the functionality as described herein. More specifically but not by way of limitation, the support member 10 could be substantially rectangular, square or other polygonal shape.

The support member 10 further includes two ends 12. Superposed substantially to each end 12 of the support member 10 is a tip 15. The tip 15 is secured proximate each end 12 by suitable mechanical or chemical methods. The tip 15 is manufactured from conventional materials that are absorbent and soft, with the ability to retain an applied solution, such as but not limited to cotton. It is contemplated within the scope of the present invention that the tip 15 has substantially disposed thereon a conventional cleaning solution such as but not limited to isopropyl alcohol or hydrogen peroxide. The tip 15 has substantially disposed thereon an appropriate amount of cleaning solution to allow a user to clean a desired area without the need for additional application of a cleaning solution to the hygienic applicator 100. While no specific size of the tip 15 is required, good results have been achieved with a tip 15 that is approximately one-half inch in length. Those skilled in the art will recognize that a tip 15 need not be present on both ends 12 of the support member 10. A tip 15 could be present only on one end 12 of the support member 10 and still achieve the desired functionality of the hygienic applicator 100 as described herein.

The hygienic applicator 100 is substantially enclosed in a container 20. The container 20 is configured in a substantially planar manner and is generally rectangular in shape. The container 20 is manufactured from a substantially flexible and non-permeable material such as but not limited to plastic or foil. The container 20 functions to prevent the evaporation of the cleaning solution that is present on the tip 15. Those skilled in the art will recognize that the container 20 could be numerous different shapes in addition to the shape as shown in the drawings submitted herewith. The container 20 is comprised of two layers 25 that are substantially the same size. The layers 25 are secured to each other by suitable mechanical or chemical methods. The layers 25 are joined to form a cavity 35 therebetween. The cavity 35 is substantially hollow and elongated in shape and is of suitable size to accommodate a single hygienic applicator 100 therein.

Proximate one end 37 of the container 20 is an opening mechanism 40. The opening mechanism 40 functions to allow a user to easily open the container 20 and gain access to the hygienic applicator 100 disposed within the cavity 35. It is contemplated within the scope of the present invention that the opening mechanism 40 is a conventional perforated treated area. Those skilled in the art will recognize that there are numerous different techniques and mechanisms that could be used in place of and/or in conjunction with a perforated area in order to assist the user in opening the container 20.

A description of the operation of the present invention is as follows. In use, a user will open the container 20 using the opening mechanism 40 to access the cavity 35 having the tips 15 of hygienic applicator 100 pre-saturated with cleaning solution disposed therein. The user then applies the cleaning solution via tips 15 to a desired area such as but not limited to the umbilical cord remnants of a newborn. The user then properly disposes of the container 20 and the hygienic applicator 100.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

1. An applicator comprising:
an elongated support member having a first end and a second end;
at least one absorbent tip disposed on said first end of said elongated support member, said at least one absorbent tip having a solution absorbed therein; and wherein said absorbent tip is comprised of cotton:
a container, said container being comprised of two plastic layers that are substantially rectangular in shape, said container further being flexible and non-permeable, said container being configured to substantially enclose said support member and said at least one absorbent tip for storage of the applicator prior to use by securing said two plastic layers together by suitable mechanical or chemical methods.

2. The applicator as recited in claim 1, wherein said support member is generally cylindrical in shape.

3. The applicator recited in claim 2, wherein said support member being at least one of plastic, wood and cardboard.

4. (canceled)

5. The applicator recited in claim 4, and further including at least a second absorbent tip disposed on said second end of said elongated support member.

6. (canceled)

7. The applicator as recited in claim 6, wherein said container further includes an opening mechanism, said opening mechanism for providing access to said container having said applicator disposed therein.

8. A hygienic applicator for cleaning various areas of a human body comprising:
a support member, said support member being substantially cylindrical in shape, said support member having first and second ends, said support member for providing structural support for said hygienic applicator; and
a first tip and a second tip, said first tip being proximate said first end and said second tip being proximate said second end, each of said first and second tips including an absorbent material for retaining cleaning solution; and wherein said first and second tins include a cleaning solution comprised of one of isopropyl alcohol or hydrogen peroxide: and wherein said absorbent material is comprised of cotton.

9. The hygienic applicator as recited in claim 8, and further including a container, said container having two layers proximate each other and forming a cavity therebetween, said container being substantially rectangular in shape and having two ends, said cavity for substantially enclosing said support member and said first and second absorbent tips in said cavity.

10. The hygienic applicator as recited in claim 9, and further including an opening mechanism, said opening mechanism proximate one end of said container, said opening mechanism for providing access to said cavity.

11. The hygienic applicator as recited in claim 10, wherein said support member includes at least one of wood, plastic or cardboard.

12. (canceled)

13. (canceled)

14. (canceled)

15. A hygienic applicator for cleaning the umbilical cord area of a newborn comprising:
a support member, said support members being substantially cylindrical in shape, said support member having two ends, said support member being manufactured from a resilient material, said support member for providing structural support for said hygienic applicator;
two tips with a single tip being proximate each of said ends of said-support member, each of said tips being manufactured from an absorbent material comprised of cotton, said tips for retaining of and subsequent application of a cleaning solutions, wherein said cleaning solution is comprised of isopropyl alcohol or hydrogen peroxide; and
a container, said container being substantially rectangular in shape, said container further having two layers forming a cavity therebetween with said support member being disposed within said cavity prior to use.

16. The hygienic applicator as recited in claim 15, wherein said container configured with an opening mechanism.

17. (canceled)

18. (canceled)

19. (canceled)

20. The hygienic applicator as recited in claim 15 wherein said support member is comprised of at least one of wood, plastic or cardboard.
